(19) United States
(12) Patent Application Publication
(10) Pub. No.: US 2021/0100503 A1
(43) Pub. Date: Apr. 8, 2021

(54) Sensor Fastener

(71) Applicant: Google LLC, Mountain View, CA (US)

(72) Inventors: Alex Olwal, Stockholm (SE); Artem Dementyev, Somerville, MA (US)

(21) Appl. No.: 17/063,098

(22) Filed: Oct. 5, 2020

Related U.S. Application Data
(60) Provisional application No. 62/911,124, filed on Oct. 4, 2019.

(51) Int. Cl. A61B 5/00

(52) U.S. Cl. CPC A61B 5/683; A61B 2562/166; A61B 2562/0219; A61B 5/6802

(57) ABSTRACT

One example aspect of the present disclosure is directed to a sensor fastener that includes at least one attachment member configured to attach the sensor fastener to an interactive object, a housing physically coupled to the at least one attachment member, an inertial measurement unit positioned within the housing, and processing circuitry positioned within the housing and communicatively coupled to the inertial measurement unit.

### RELATED APPLICATIONS

This application is based on and claims priority to U.S. Provisional Patent Application Number 62/911,124, titled “Sensor Fastener,” filed on October 4, 2019 which is hereby incorporated by reference herein in its entirety.

### FIELD

The present disclosure relates generally to wearable devices including sensors.

### BACKGROUND

Interactive objects such as wearable devices integrate electronics into a garment, accessory, container or other article worn or carried by a user. Many wearable devices include various types of sensors integrated within the wearable device to measure attributes associated with a user of the wearable device. By way of example, wearable devices may include heart-rate sensors that measure a heart-rate of a user or motion sensors such as accelerometers, gyroscopes, etc. that measure distances, velocities, steps or other movements associated with a user. Some wearable objects may include sensing lines such as conductive threads or conductive lines incorporated into the interactive object to form a sensor such as a capacitive touch sensor that is configured to detect touch input. Wearable devices may also include conductive lines for other purposes, such as for strain sensors using conductive threads and for visual interfaces using line optics. A wearable device can process touch inputs, motion inputs, or other inputs received by conductive lines, motions sensors, and other sensors to generate input data that is useable to initiate functionality locally at the interactive object or at various remote devices that are wirelessly coupled to the wearable device.

While various techniques for integrating electronics and wearable interfaces into garments and other interactive objects have been proposed, many existing solutions have proven less than ideal. For example, it can be challenging to augment garments with electronics when the use of the garment over its lifetime is considered. Manufacturing constraints, end-user customizations, and maintenance may need to be considered. Some designs utilize sewn connections to attach electronics to garments. Such designs may require skill and time to implement into a workable garment. In some cases, the integration of electronics into garments results in stiff areas due to a need to accommodate rigid components. Accordingly, there remains a need for systems and methods that provide for the integration of electronics into garments and other objects to form wearable devices.

### SUMMARY

Aspects and advantages of embodiments of the present disclosure will be set forth in part in the following description, or may be learned from the description, or may be learned through practice of the embodiments.

One example aspect of the present disclosure is directed to a sensor fastener including a first fastener component, a second fastener component, and a third fastener component releasably coupled with the first fastener component to releasably fasten at least two portions of an interactive object to one another. The second fastener component includes a housing, an inertial measurement unit disposed within the housing, processing circuitry disposed within the housing and communicatively coupled to the inertial measurement unit, and at least one attachment member that physically couples the housing to at least the first fastener component.

Another example aspect of the present disclosure is directed to a sensor fastener including a post, a stud configured to pass through a flexible substrate of an interactive object and physically attach to the post, a socket releasably and physically coupled to the post, and a cap. The cap includes a housing, an inertial measurement unit disposed within the housing, processing circuitry disposed within the housing and communicatively coupled to the inertial measurement unit, and at least one attachment member configured to pass through the flexible substrate and physically couple the housing to the socket of the sensor fastener.

Yet another example aspect of the present disclosure is directed to an interactive object, including at least one substrate at least one sensor fastener physically and removably coupled to the at least one substrate. The sensor fastener comprises a post, a stud that passes through the at least one substrate and physically couples to the post, a socket releasably and physically coupled to the post, and a cap. The cap includes a housing, an inertial measurement unit disposed within the housing, processing circuitry disposed within the housing and communicatively coupled to the inertial measurement unit, and at least one attachment member that passes through the at least one substrate and physically couples the housing to the socket.

Other example aspects of the present disclosure are directed to systems, apparatus, computer program products (such as tangible, non-transitory computer-readable media but also such as software which is downloadable over a communications network without necessarily being stored in non-transitory form), user interfaces, memory devices, and electronic devices for processing sensor data.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

Detailed discussion of embodiments directed to one of ordinary skill in the art are set forth in the specification, which makes reference to the appended figures, in which:

FIGS. 1A-1D depict examples of sensor fasteners in accordance with example embodiments of the present disclosure;

FIG. 2A-2B depicts parts of an example sensor fastener in accordance with example embodiments of the present disclosure;

FIGS. 3A-3C depict an example sensor fastener including a printed circuit board in accordance with example embodiments of the present disclosure;

FIGS. 4A-C depict an example of an attachment of an example sensor fastener to fabric in accordance with example embodiments of the present disclosure;

FIG. 5A-5D depicts an example mechanical enclosure design of a sensor fastener in accordance with example embodiments of the present disclosure;

FIG. 6 depicts a system diagram of an example wireless network including one or more example sensor fasteners in accordance with example embodiments of the present disclosure;

FIG. 7 depicts example data from a gyroscope during left and right rotation in accordance with example embodiments of the present disclosure;

FIG. 8 depicts an example of accelerometer data during a tap in accordance with example embodiments of the present disclosure;

FIG. 9A depicts an example of capacitive touch sensing using a sensor fastener in accordance with example embodiments of the present disclosure;

FIG. 9B depicts an example of a sensor fastener including a capacitive touch sensor in accordance with example embodiments of the present disclosure;

FIG. 10 depicts a gyroscope angle in comparison to a reference angle in accordance with example embodiments of the present disclosure;

FIG. 11 depicts example sensor fasteners attached to the arm and the torso of a user for motion tracking in accordance with example embodiments of the present disclosure;

FIG. 12 depicts an example computing environment in accordance with example embodiments of the present disclosure; and

FIG. 13 depicts various components of an example computing system that can be implemented as any type of client, server, and/or computing device as described herein.

### DETAILED DESCRIPTION

Reference now will be made in detail to embodiments, one or more examples of which are illustrated in the drawings. Each example is provided by way of explanation of the embodiments, not limitation of the present disclosure. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations.

Adding electronics to textiles can be time-consuming and require technical expertise. Systems and methods in accordance with example embodiments are directed to sensor fasteners, which in example implementations can include low-power wireless sensor nodes that seamlessly integrate into caps or other fastener components of snaps or other fasteners. Sensor fasteners in example embodiments provide a technique to quickly and intuitively augment any location on clothing or other objects with sensing capabilities. Sensor fasteners can securely attach and detach from ubiquitous commercial snap fasteners. Using inertial measurement units, the sensor fasteners can detect tap and rotation gestures, as well as track body motion. In some examples, the power consumption is optimized for the sensor fasteners to work continuously for long periods of time, and even longer periods of times in a standby mode such as a capacitive touch standby mode. Various applications are provided in which the sensor fasteners can be used as gestural interfaces for various application controllers, cursor control, motion tracking suit, or other applications. Sensor fasteners as described herein can be attached to existing garments or other objects in short periods of time, which can be similar to attaching off-the-shelf snaps or other fasteners. Additionally, the gestures performed with the sensor fastener are easy to learn and perform. Sensor fasteners can allow anyone to effortlessly add sophisticated sensing capacities to ubiquitous snap fasteners.

Recent advances in materials and electronics have inspired a surge in the development of more seamless and ubiquitous wearable devices. In particular, the potential for integrating electronics and wearable interfaces into clothing has been shown particularly promising. It is, however, challenging to augment clothing with electronics, given considerations across the lifetime of a garment, including manufacturing constraints, end-user customization, and maintenance.

Some examples of the integration of electronics with wearable devices include yarn-level innovations for integration at manufacturing time that are compatible with existing textile processes. These projects have the potential to enable interactive textiles at scale with predefined capabilities in the garments. These efforts can be complemented by leveraging traditional craft techniques for customization and modifications of existing textiles. The integration with electronics, however, tends to be done through sewn connections, where adding and removing functionality may require specialist skills and time.

Many garments already employ small, rigid parts for both functional and decorative purposes, such as buttons or fasteners in places where the fabric supports reconfigurability, for opening, closing, folding, or changing its length. As described herein, a sensor fastener can leverage advances in miniaturized electronics to augment such buttons or other fasteners with interactive capabilities without manufacturing dependencies. Various fastener categories can be utilized in accordance with the disclosed technology. For example, one category of fasteners includes the button category, which may include snap fasteners. Snap fasteners in accordance with example embodiments are widely available and allow integration into clothing with limited knowledge and tools. They also provide minimal constraints for attachment and removal, which can be beneficial for flexibility with customization and maintenance. It will be appreciated that a snap is provided as one example of a button category and that sensor fasteners as described herein can be implemented in various ways in example implementations.

In some examples, sensor fasteners as described herein may be inexpensive and incorporate available interactive clothing fasteners for purchase from textile suppliers or in fabric stores. This can allow individuals, as well as manufacturers, to augment garments with wireless electronics and sensors easily. Thus, in addition to standalone operation, the described approach may also seamlessly co-exist with electronics for yarn-level integrations, as well as with craft-based techniques.

In some examples, a sensor fastener as described herein can enable many applications that can leverage touch- and motion-sensing, and ubiquitous sensor networks, since they can be added anywhere on clothing, textiles or other objects. For example, activity tracking or motion sensing during sports activities can be implicitly captured by sensor fasteners on the clothing, as an alternative to wearing smartwatches or hook and loop-strapped sensor nodes. Instead of using separate remote controls or external sensors, sensor fasteners as described herein can be used to add gestural control on clothing, for example, on shirt cuffs to control heads-up displays, an in-car navigation system, or a slide presentation.

In accordance with some example embodiments, a generic snap fastener can be augmented with miniaturized electronics that perform on-device gesture recognition, wireless connectivity, motion tracking with sensor fusion, and touch sensing, with the potential for all-day battery life under episodic use. In accordance with some examples, a method to design such devices in order to meet stringent power and size requirements is provided. A technical evaluation can demonstrate and characterize the hardware described herein. Multiple applications are provided that show the capabilities of sensor fasteners, as illustrated by a discussion of results from a qualitative user study to evaluate the interactions and applications enabled by sensor fasteners.

In accordance with example embodiments, a miniaturized wireless sensor node implementation is provided that integrates into snap button caps. A technical evaluation and characterization of power, cost and IMU performance is described. Examples of various implementations of sensor fasteners and interactions are provided, such as body motion tracking, and keyboard input using a chorded keyboard embedded in a shirt cuff. A qualitative user evaluation demonstrates the usefulness of various sensor fasteners and interactions. In some example embodiments low-power firmware is provided that uses IMU data to detect gestures, such as tapping and rotation. A sensor fastener in example embodiments can have a long continuous battery life and an even longer battery life in a capacitive touch standby mode.

In some examples, a sensor fastener can include miniaturized modules that can be used in various form factors. A sensor fastener can provide unique capabilities enabled through a snap fastener integration, which opens up new possibilities for seamless integration with the most common clothing and accessories.

According to some example embodiments, an extension of opportunities demonstrated through worn or held device form factors is provided with a sensor fastener approach, where motion-based sensing can be embedded at manufacturing time for discrete and direct integration on the garments themselves, through scalable augmentation of existing snap fastener components.

In some examples, sensor fasteners can enable a body sensor network using Bluetooth or other communication to coordinate and collect data from a cluster of distributed modules. Such designs can be flexible with location and placement to enable following of the existing style or design of a garment.

In example embodiments, interactive textiles at scale are enabled. A sensor fastener approach can be a complementary approach where embedding miniaturized electronics in existing mass-producible snap fastener components provides additional options for interactive textiles to product designers and engineers.

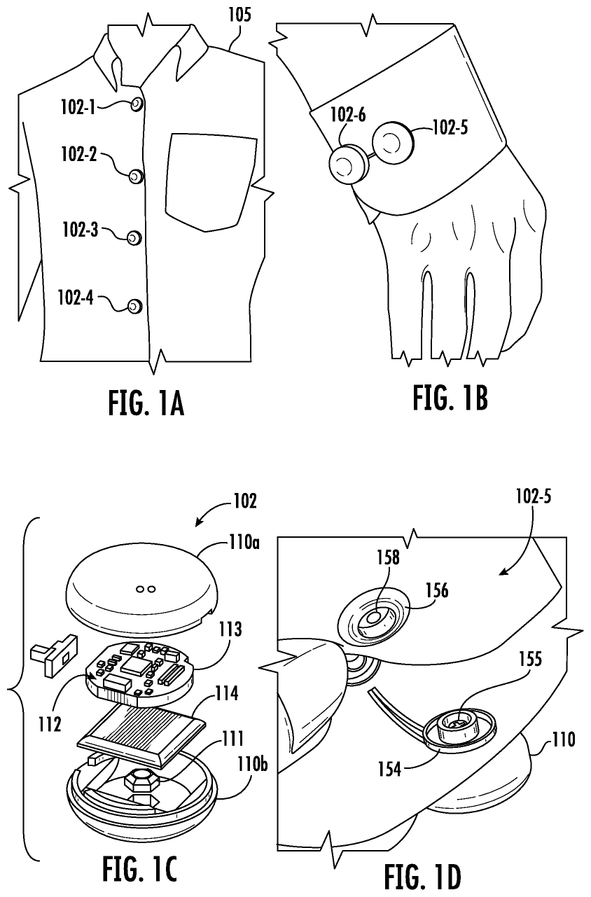

FIGS. 1A-1D illustrate examples of sensor fasteners 102 in accordance with example embodiments of the present disclosure. In the particularly described example depicted in FIG. 1A, sensor fasteners 102-1, 102-2, 102-3, and 102-4 can be used to replace the original buttons, snaps, or other fasteners that are used to close the front of a shirt 105. In other examples, a sensor fastener can be used to form the original button, snap, or other fastener on a shirt. A sensor fastener such as a sensor snap can embed miniaturized electronics into a generic snap or other fasteners. This makes it possible to augment garments with wireless electronics that enable on-device gesture recognition, wireless connectivity, motion tracking with sensor fusion, and touch sensing. As shown in FIG. 1A, sensor fasteners 102-1, 102-2, 102-3, and 102-4 can replace buttons on a shirt. As shown in FIG. 1B, sensor fasteners 102-5, 102-6 can be used as cufflinks or as another fastener that can close the cuff opening of a garment. Each sensor fastener includes at least one fastening component physically coupled to a first portion of the shirt 105 and at least one fastening component coupled to a second portion of the shirt. In this manner, the sensor fastener can function as a traditional mechanical fastener to releasably couple two portions of a garment, garment accessory, garment container, or other interactive object together.

As shown in FIG. 1C, an exploded CAD model of a sensor fastener 102 is depicted. The electronics 112 and the battery 114 can be contained inside a housing 110, also referred to as a case. The housing may include a lid 110a and base 110b that can be coupled to one another to enclose electronics 112 of circuit board 113 and battery 114. Base 110b can be attached to the fabric substrate of shirt 105 using at least one attachment member such as a screw. Electronics 112 can include one or more integrated circuits or other components formed on a printed circuit board 113.

As shown in FIG. 1D, integration with off-the-shelf plastic snap fasteners can be provided in some examples. The sensor fastener can include a plurality of fastener components that physically couple the sensor fastener to an interactive object or a user of an interactive object. For example, the fastener components can be physically coupled or otherwise attached to a flexible substrate (e.g., textile substrate) of a garment, garment accessory, or garment container. Two or more of the fastener components can releasably couple with one another to form a mechanical fastener for the interactive component. One or more of the fastener components can include electronics including one or more sensors to form the sensor fastener. The sensor fastener 102-5 can include a cap 152 that is formed by housing 110 which contains electronics 112 on a printed circuit board 113. Additionally, sensor fastener 102-5 can additional fastener components including a socket 154, a stud 158, and a post 156 in example embodiments. Socket 154 can be physically attached to housing 110 using a screw 155 or other attachment member. Screw 155 can pass through an opening in the socket 154, through the flexible substrate (e.g., textile substrate) of shirt 105, and physically couple to the housing 110. Sensor fastener 102 can include a receiving member 111 disposed on the inside of base 110b to receive screw 155. The receiving member 111 can be a nut or other threaded component in example embodiments. In this manner, the screw physically couples the socket 154 to the shirt 105 by coupling the housing 110 to socket 154 on opposite sides of the textile substrate. This provides secure attachment of the sensor fastener to the shirt 105. In an alternate example, socket 154, stud 158, and/or post 156 may include all or portion of electronics 112 and/or battery 114. Socket 154 and post 156 may releasably snap together and thereby releasably fasten the end portions of a cuff of shirt 105.

In example embodiments, the described design can provide compatibility with off-the-shelf clothing. The design can require minimum redesign of current technology to allow seamless integration and installation into existing clothing. The design can provide quick attachment and detachment. For example, the sensor fastener 102 can be quickly added to and removed from clothing. The design can provide low power as power is often a constraint for small wearable devices. For example, the sensor fastener in some examples can have a battery life of at least a few hours and can be rechargeable. The design can provide a small and lightweight footprint, so the sensor fasteners are not obtrusive during everyday wear. By way of example, commercial snap fasteners are typically 9-15 mm in diameter. The design can also provide a lightweight solution. For example, the sensor fasteners can weigh no more than a few grams in some examples, which is comparable with off-the-shelf metal snaps (e.g., up to 1g). The design can provide on-device sensing, computation and communication. This allows multiple sensor fasteners to function independently, for easy integration and also as building blocks for complex and distributed wearable systems.

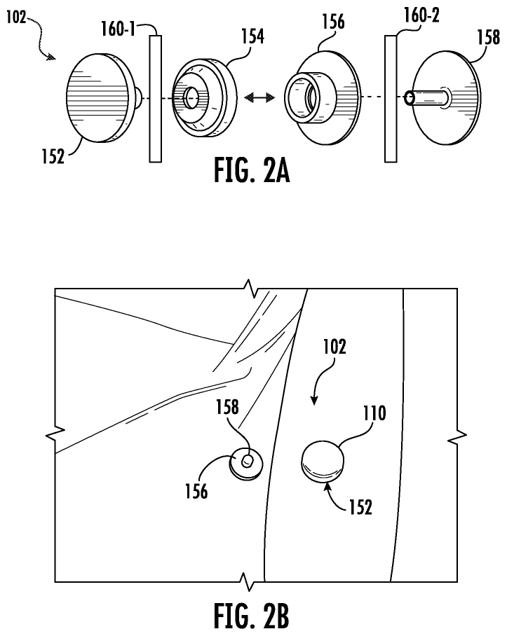

FIGS. 2A-2B depict parts of a sensor fastener in accordance with example embodiments of the present disclosure. FIG. 2A depicts various parts of a standard snap fastener 150 which can be used to implement a sensor fastener. Snap fastener 150 includes a plurality of fastener components, including a cap, socket, stud, and post. As illustrated, a first portion 160-1 of a fabric substrate is sandwiched between a cap 152 and a socket 154. Another portion 160-2 of the fabric substrate is sandwiched between a post 156 and a stud 158. For attachment, the post 156 snaps into the socket 154. In some examples, a sensor fastener can be formed by modifying a cap only. For instance, a sensor fastener 102 may include electronics 112 and/or battery 114 formed within cap 152 in example embodiments. FIG. 2B depicts an example of a metal snap fastener on a winter jacket, illustrating a cap 152, post 156, and stud 158

Various methods may be used to attach electronics to a body or interactive object. A review of advantages and disadvantages of potential body and object attachment methods is provided. Integrating technology directly into textiles can enable more seamless designs, but can be difficult to customize due to the challenges of bridging soft and hard components in the textile-electronics interface. Straps and hook and loop type fasteners (e.g., velcro) can be used to attach electronics to clothing or body quickly, but may not integrate into clothing. Buttons clothing fasteners with small electronics may integrate well into clothing but may need to be permanently sewn. Commonly, such buttons are thin so they can be pushed through a buttonhole and contain four holes for sewing. Their small size and thread holes in the middle can make it hard to include electronics.

As shown in FIG. 2A, common snap-buttons contain four parts referred to as fastener components. They are typically made from plastic or metal. To connect two portions 160-1 and 160-2, a cap 152 and socket 154 on one side or portion of the fabric snap into a post 156 and stud 158 using a friction-based snap fit. In such embodiments, the cap/socket releasably couples to the post/stud, thereby releasably fastening the two portions of the fabric together, such as to close a cuff or the front of a shirt. In accordance with example embodiments, the cap part of a fastener can be modified to form a sensor fastener. The cap 152 is the part facing the outside so that it can be used for access for gestures in some examples. In such examples, the cap and socket together can form attachment members for the sensor fastener. Specifically, the connection between the cap and socket can provide an attachment of the sensor fastener to the textile substrate. In an alternate example, the post and/or stud part of a fastener can be modified to form a sensor fastener. In such examples, the post and stud together can form attachment members for the sensor fastener. Specifically, the connection between the post and stud can provide an attachment of the sensor fastener to the textile substrate.

In accordance with example embodiments, a snap fastener attachment can avoid damage to the sensitive circuit (e.g., electronics 112 and/or PCB 113) during assembly, creating a removable connection, and enable easy and quick installation. Various methods can be used to attach snap fasteners to the fabric. In one example, a sewn connection is provided. Snap fasteners can be sewn to the fabric. The sensor fastener can be removed by cutting the threads.

In another example, a crimping tool can be used. Another approach is to use a specially designed crimping tool. The fabric can be sandwiched between two parts of the snap fastener, and the crimping tool apply a force to deform a specially designed area of the snap to make a permanent connection. The specially designed area that is deformed can form an attachment member. The crimping tool can produce large forces on the snap fastener. In some examples, the sensor fastener is constructed to withstand these pressures for electronics and 3D-printed parts. In some examples, buttons can be removed for reconfigurability or washing.

In yet another example, a screw can be used as an attachment member. Two fastener components of a sensor fastener can be secured with a screw, which can fit many use cases well. The screw can be removable and does not damage the electronics. In example embodiment, the screw may attach a base 110b portion of a sensor fastener housing to a textile substrate by passing through the textile substrate and physically coupling with a socket on the opposite side of the fabric.

Sensor fasteners can be attached to garments using various methods. To attach the sensor fastener to the fabric in some examples, the following steps can be performed. First, a hole can be punched in the fabric. This can be done by simply pushing the sharp end of an off-the-shelf cap through the fabric or with a die hole punch for thick fabric or leather. Second, the sensor fastener cap and off-the-shelf socket can be placed on opposite sides of the hole. A screw can be added on the socket side to secure the cap and socket. The post and stud side can be attached in a standard way using a fabric crimp tool in some examples. The fabric crimp tool can deform a portion of the stud and/or post, thereby forming an attachment member between the two fastener components.

Detaching commercial crimp snaps can be difficult in some instances and result in destroying the snap with snippers (e.g., sewing scissors) or pliers. In accordance with example embodiments of the present disclosure, a sensor fastener can be quickly separated by removing a screw or other attachment member. The removal process may leave a small screw hole in some instances, but it can become less noticeable over time. The hole can be formed mainly by the displacement of the fibers of the fabric, which over time tend to return to their original position.

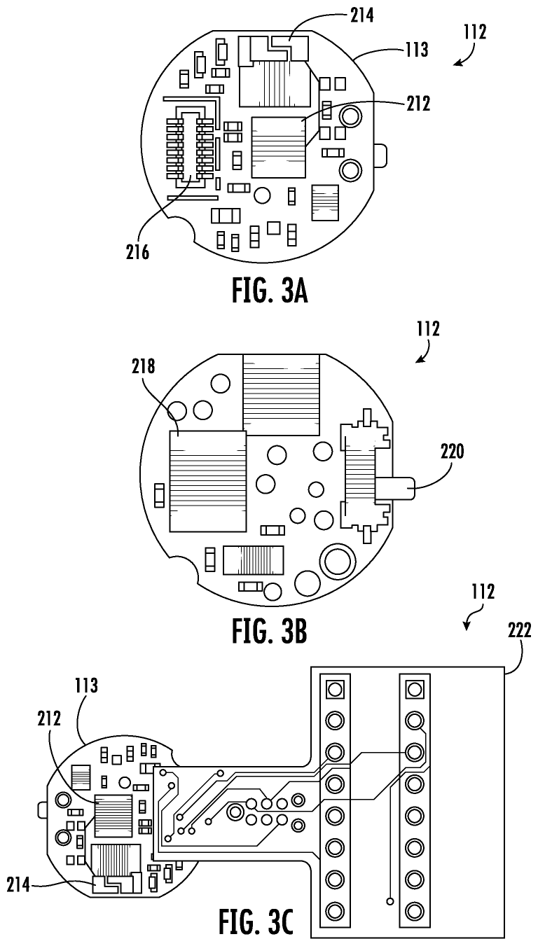

FIGS. 3A-3C depict an example of a sensor fastener 102 including electronics 112 formed on a 12 mm diameter custom-made circuit board 113. Other sizes can be used. An example printed circuit board 113 (PCB) for a sensor fastener is shown. FIG. 3A depicts a top side view illustrating a microcontroller 212, antenna 214, and 20-pin port 216. FIG. 3B depicts a bottom side view illustrating an IMU 218 and an on/off switch 220. FIG. 3C depicts a programming and charging connector for a 20-pin port.

The sensor fastener can include a controller 212 such as a microcontroller (e.g., a nRF52832 microcontroller available from Nordic Semiconductors) to perform processing for the sensor fastener. A microcontroller chip can include one or more processors such as one or more processing cores (e.g., an ARM Cortex M4F), as well as a radio such as a wireless 2.4 GHz radio in example embodiments coupled to one or more antenna 214. The sensor fastener can employ a Bluetooth Low Energy (BLE) protocol in example embodiments. The Bluetooth radio can connect to a mobile phone or a computer and has low power consumption. In some examples, the microcontroller provides a small size and integrated Bluetooth enabled radio.

The sensor fastener can additionally include an inertial measurement unit 218. In some examples, the inertial measurement unit is a 9-axis inertial measurement unit (IMU) (e.g., BNO055 available from Bosch). The inertial measurement unit can contain onboard sensor fusion functionality for absolute orientation in some examples. The sensor fastener can include a power source. In some examples, a, 10 mAh lithium polymer battery can be used (e.g., PGEB201212, General Electronics Battery Co.) to power the device. Lithium polymer batteries can satisfy various energy density and size requirements. Also, the circuit board can include a 20-pin or other size connector or port 216 for charging, programming and as an expansion port for additional functionality. In some examples as shown at C), an adapter 222 provides access to battery pins that are used to charge with an external charger and programming pins. In some examples, a printed circuit board can include a standard port such as a micro USB. In another example, the printed circuit board may be smaller than a standard port, such as micro USB.

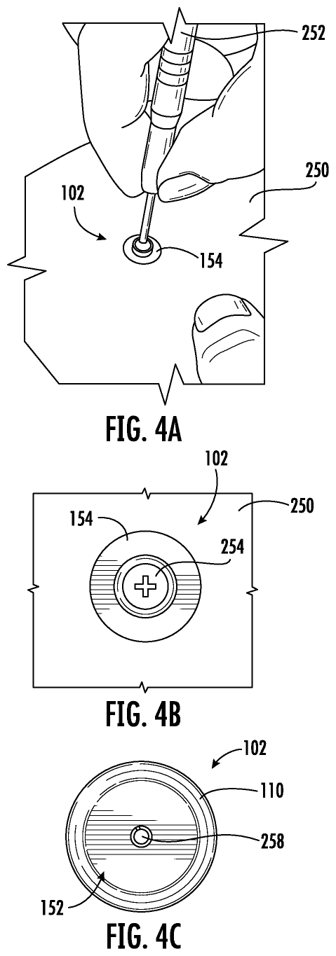

FIGS. 4A-4C depict an example of an attachment of a sensor fastener to a fabric 250 textile substrate. FIG. 4A depicts a user using a screw-driver 252 to attach the housing 110 of the sensor fastener 102 to the plastic socket 154 (e.g., off-the-shelf component) using a screw 254. The housing is on the other side of the fabric 250 and can form the cap 152 for the sensor fastener assembly. In FIG. 4B, the socket 154 is depicted after it is attached to the sensor fastener with screw 254. FIG. 4C depicts the backside of sensor fastener 102, showing an opening 256 such as a hex nut screw hole through housing 110. The hex nut screw hole may be threaded in example embodiments or otherwise configured to receive and couple to screw 254. In example embodiments, housing 110 including the electrical components of the sensor fastener can form a cap 152 as shown in FIG. 2A.

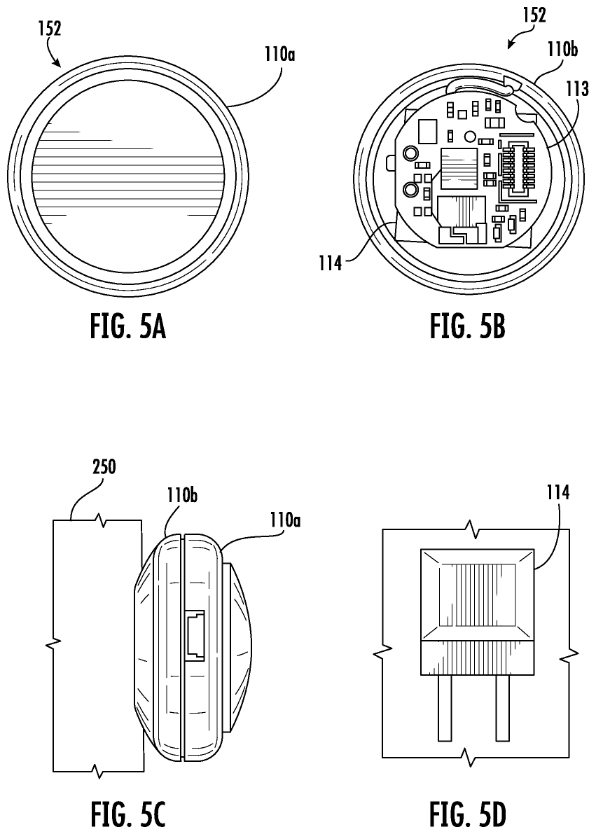

Various mechanical parts can be used to fabricate a sensor fastener in accordance with example embodiments of the present disclosure. In some examples, a sensor fastener can include a custom enclosure for the electronics. In some examples, a sensor fastener can replace the cap of a snap fastener. An example enclosure is shown in FIG. 5. In some examples, the parts can be 3D printed using a 3D printer (e.g., SLA printer, Form 2 and Black V4 resin, Formlabs). The designed enclosure can fit with standard snap fasteners and remain robust and small in some examples. The snap button can interface with a socket using one or more attachment members. For instance, the snap button can interface with a socket using an M2.0 hex nut and a 4 mm long screw in some examples. A flat head screw can be used so it does not interfere during snapping. An M2.0 screw can provide a large size that can fit with standard plastic snaps. In one particular example, the total weight of a sensor fastener can be about 2.4 grams (e.g., electronics: 0.6 g, battery: 0.6 g, mechanical parts: 1.2 g). Sensor fasteners may have different weights in different implementations. As a comparison, off-the-shelf plastic caps can weigh about 0.1 grams and metal caps can weigh about 0.8 grams. Other types of attachment members can be used.

FIG. 5 depicts an example of a 3D printed mechanical enclosure design for sensor fastener 102. FIG. 5A depicts the cap 152 of a sensor fastener 102 with a lid 110a attached. FIG. 5B depicts the cap 152 of the sensor fastener without the lid 110a, showing the printed circuit board 113 (PCB) and the battery 114 behind the PCB 113. FIG. 5C depicts the enclosure from the side including lid 110a attached to base 110b. FIG. 5D depicts a 10 mAh lithium polymer battery 114 (e.g., PGEB201212, General Electronics Battery Co.) with a thickness of 2 mm. It will be appreciated that different mechanical designs may be used.

Multiple sensor fasteners can be used to form a wireless sensor network. For example, sensor fasteners can be used to form a simple sensor network of Bluetooth nodes. In some examples, a star topology can be used with one central node and sensor fasteners provided as peripheral nodes. After turning on, the sensor fasteners can start advertising their name on the BLE network. The central node can continuously scan for more sensor fasteners, even if some are already connected. The central node can automatically attempt to connect to new sensor fasteners that are discovered.

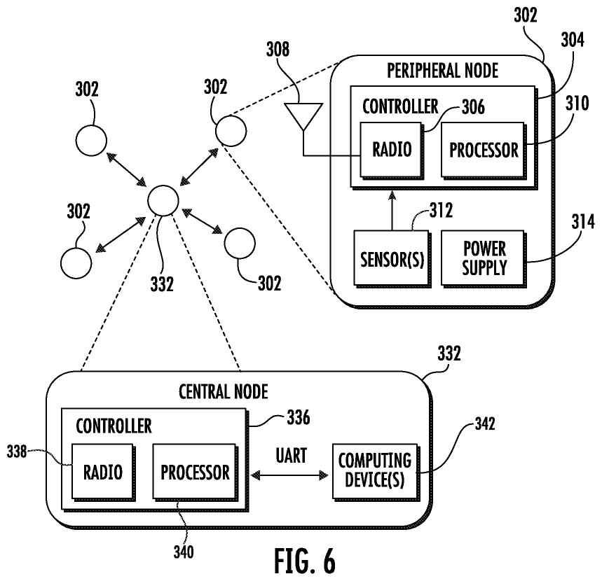

FIG. 6 depicts a system diagram of an example wireless network 300 including one or more sensor fasteners in accordance with example embodiments of the present disclosure. In this example, the sensor fasteners are peripheral nodes 302 (e.g., peripheral BLE nodes), which connect to one central node 330.

The peripheral nodes can include a controller 304 (e.g., microcontroller) having one or more processors 310 and a radio 306, such as a 2.4 Ghz Bluetooth radio, connected to an antenna 308. The peripheral nodes 302 can include one or more sensors 312 such as an IMU (e.g., 9-axis IMU) and a power supply 314 such as a battery. The central node 332 can include a controller 336 having one or more processors 340 and a radio 338, such as a 2.4 Ghz Bluetooth radio. The controller 336 can communicate with one or more computing devices 342 using a Universal Asynchronous Receiver/Transmitter (UART).

In some examples, a BLE network utilizes a central node 332 to manage the connections. Many devices can act as a central node 332. By way of example, a laptop, smart phone, or a development board (e.g., an NRF52840 development board (NRF52840 DK, Nordic)) can be used as central nodes. Other devices can be used for a central node 332. Technical evaluations and development can be done with a development board as a central node as it can provide full chip-level access to BLE and debug interfaces, which may not be as conveniently accessible when using a built-in Bluetooth of a laptop or smart phone. For some applications, a smart phone or the Bluetooth of a laptop can be used as the central node.

To conserve energy, sensor fasteners can be configured to only send data over Bluetooth when an event, such as a tap, is detected. The central node can be always listening for new data. For continuous gestures, such as rotation, the sensor fasteners can be capable of sending data continuously at a rate of 50 Hz. Other rates can be used. The continuous mode is only used sporadically in some examples.

In accordance with example embodiments, gesture detection can be done on-board the sensor fastener. Streaming raw data may not be energy efficient as wireless transmission can be power intensive. Accordingly, data can be sent only when gestures are detected in some examples.

In some examples, a sensor fastener can provide for tap gesture detection. A tap gesture can be detected using the accelerometer to detect when the sensor fastener is tapped. In some examples, tap gesture detection can be implemented in firmware. In other examples, tap gesture detection can be built into the IMU. In some examples, a tap detection algorithm can be based on a slope detection method, which is implemented in some IMUs. The accelerometer's three axes can be sampled at 33 Hz or another frequency. To subtract the gravity, the slope of the accelerometer can be calculated at every new data point.

In accordance with example embodiments, a sensor fastener can be configured to detect a rotation gesture. A rotation gesture can use the gyroscope to detect the rotation of the sensor fastener. To obtain the rotation angle, the z-axis of the gyroscope angular velocity can be integrated at 33 Hz or another frequency. An example of the data is shown in FIG. 7. FIG. 7 depicts example data from a gyroscope during left and right rotation at 352. Raw angular velocity can be integrated to obtain rotation angle as illustrated at 354.

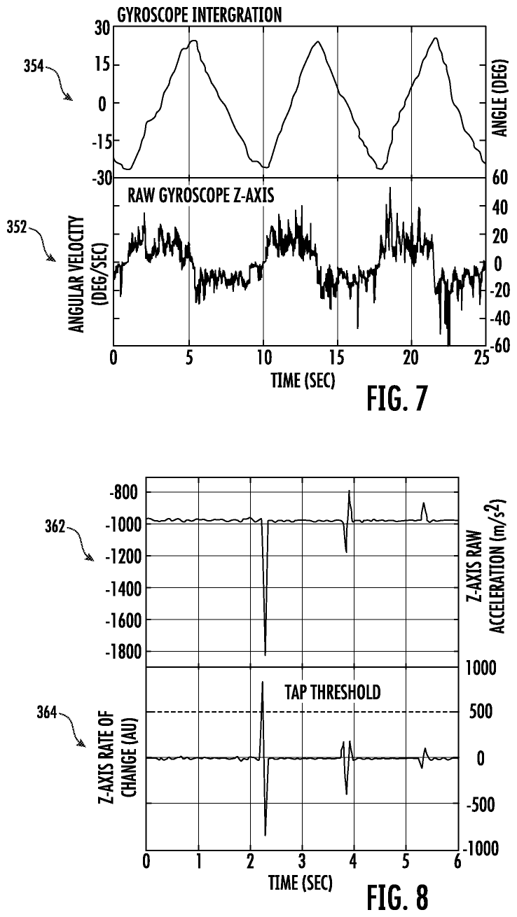

FIG. 8 depicts an example of accelerometer data during a tap. Raw acceleration data is shown at 362. The rate of change of the raw acceleration is illustrated at 364 and can be used to detect taps. When the slope reaches a threshold, a tap gesture can be registered and transmitted over Bluetooth.

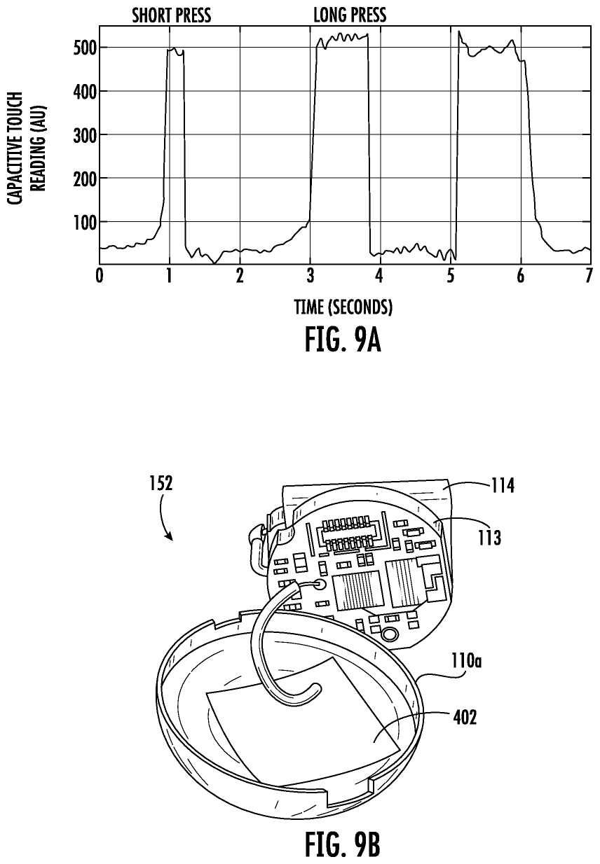

FIGS. 9A-9B depict an example of capacitive touch sensing using a sensor fastener 102 in accordance with example embodiments of the present disclosure. FIG. 9A depicts an example of capacitive touch raw data for long and short presses. FIG. 9B depicts an example of a sensor fastener that includes a capacitive touch electrode 402 on the back of the lid 110a of the housing of the sensor fastener. Specifically, FIG. 9B depicts the cap of a sensor fastener.

A touch gesture detected via capacitive touch sensing can detect when the sensor fastener is being touched. This gesture uses a capacitive sensing electrode 402 on the back of the sensor fastener lid 110a in some examples. The electrode can be made out of a plastic-backed copper foil (e.g., Pyralux AP, DuPont), cut by hand or otherwise and attached with epoxy or other means. The electrode can be soldered to a pin of a microcontroller and can use a firmware driver.

A capacitive touch gesture can be employed for wakeup. It is similar to a tap gesture but detects direct touch; therefore it can be more robust to motion artifacts. Furthermore, the touch gesture can only use the microcontroller in example embodiments, so it can save power by leaving the IMU in sleep mode. Capacitive touch sensing can be provided by adding an electrode to the device. Raw data from a capacitive touch sensor in example embodiments is shown in FIG. 9A. A simple threshold can be applied to detect touch events, which is determined experimentally after the sensor fastener is placed inside the enclosure. The electrode can be sampled every 25 ms or other intervals. No additional calibration is required for capacitive touch gestures in some examples. For instance, IMU-based gestures may not require calibration.

In some examples, sensor fasteners can provide power optimization. In some situations, energy may be extremely constrained for a sensor fastener implementation. Multiple strategies can be employed to conserve the battery. One strategy is to stay in standby mode as much as possible.

Example modes include a standby mode. The standby mode can be the lowest power mode. In the standby mode, the sensor fastener can perform two primary functions. First, to keep the BLE connection alive, BLE packets can be sent at predetermined intervals to prevent the peripheral and central clocks from drifting apart. Synchronization can be used such that the sensor fastener responds to the central node every 500 ms. Second, the capacitive touch sensor can be periodically sampled for touches. If a touch is detected, the sensor fastener can go into a gesture mode.

The gesture mode allows real-time interactions with minimum latency, but may consume more power than the standby mode. In this mode, the BLE connection interval can be less than in the standby mode, which means that packets can be exchanged more frequently. The gyroscope can be turned on to sense the rotation gestures. Also, capacitive sensing can be turned on. If no gestures are detected for 10 seconds, the device can go back to standby mode.

A motion sensing mode can be a most power-expensive mode, allowing for 3D orientation tracking with the IMU. The power consumption can be the highest in this mode since the accelerometer, gyroscope, and magnetometer are turned on. Also, the BLE connection interval can be the same as in the gesture mode or can be different. In some examples, 30-byte packets can be used. The packets can contain a unique 1-byte id, quaternions, and data deliminators.

A technical evaluation of an example implementation of a sensor fastener in accordance with example embodiments is provided to illustrate technical performance and usability.

Power consumption can be measured using a digital multimeter in the three power modes. To capture the transient current changes due to BLE transmissions, the current can be sampled at 77 Hz or another frequency, and the mean current over 5-minute intervals reported.

The power consumption in the standby mode is 2.45 mA (8.10 mW) in this particular example. The sensor fastener can last 4 hours in this mode. The current in this mode is mostly consumed by the capacitive touch sensing (1.78 mA), and the rest (0.68 mA) is used to keep the BLE connection and timers active. By turning off the capacitive sensing, the power consumption can be reduced at the expense of missing touch events. By reducing touch sensing to the minimum, the battery life can be extended to 15 hours.

The power consumption in the gesture mode may be 13.72 mA (45.28 mW) and 16.06 mA (53.00 mW) in the motion tracking mode. In those two modes, most energy (around 9 to 11 mA) was consumed by the IMU. A sensor fastener in accordance with example embodiments can be ubiquitous and provided at low cost.

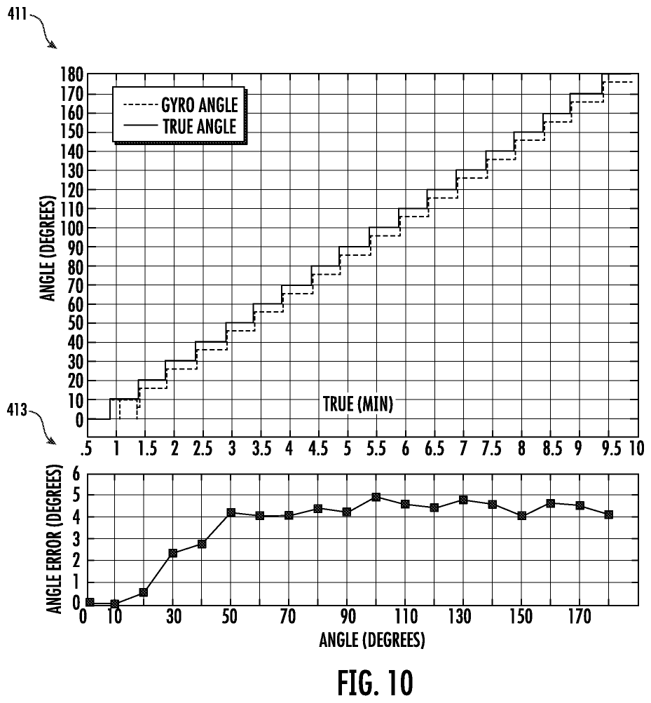

FIG. 10 depicts a gyroscope angle in comparison to a reference angle at 411. The error between the gyroscope angle and the true angle is plotted on the bottom at 413 for each angle. The accuracy of gyroscope angles can be evaluated in comparison to reference angles. The gyroscope can be moved from 0 to 180 degrees in z-axis in ten-degree increments. The gyroscope integration mean error can be about 3.53 degrees (Standard deviation:±1.62). A test can be performed for gyroscope drift. In some examples, no drift is measured over 10 minutes on a stationary device. In an exemplary simple test, a sensor fastener is able to wake up and transmit a packet over BLE in 142 out of 150 finger touch events (94.7%).

A user evaluation can provide an understanding of the usability of a sensor fastener as well as gain insight on how it will be used in the future.

In example embodiments, a sensor fastener can be used to control a computer during a presentation, interact with a smartphone to reply to a call or a text, and navigate the contents of an audiobook. A sensor fastener can be used for biosignal detection (heart and respiration rate), activity and movement logging or environmental sensing (UV exposure, temperature, humidity).

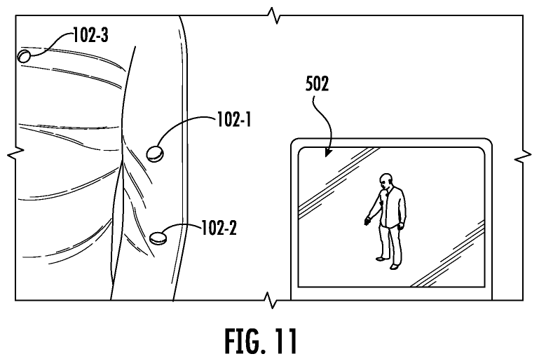

In some examples, sensor fasteners can be used for body motion tracking. FIG. 11 depicts multiple sensor fasteners 102-1, 102-2, and 102-3 attached to the arm and the torso for motion tracking in accordance with example embodiments. A resulting 3D animation 502 is shown on the computer screen.

Motion tracking can provide useful information for sports, medicine and gesture-controlled devices. Traditionally, optical motion tracking is done by tracking reflective markers with multiple cameras. Using 9-axis IMUs attached to different body parts, motion tracking can be done without external cameras. Currently, the IMU approach is still cumbersome and requires a special suit equipped with IMUs. Sensor fasteners can be added to off-the-shelf clothing to enable motion tracking on demand. A sensor fastener can be attached to the arm, forearm and torso and quaternion orientation data is continuously sent to the computer. To visualize the data, the quaternions can be received in Blender, and used to control the limbs of a virtual animated character.

In some examples, a sensor fastener can be charged using a contactless or magnetic connector. Charging can be done without the need to remove the top part of the cap in some examples. This can allow the sensor fastener to be water sealed and washable. Inductive charging can be used to charge a sensor fastener wirelessly. An inductive charging coil (Qi certified) can be used that can fit inside the small cap. Alternatively, the snap connector can be made conductive so that it can snap to a charger. In some examples, special pins can be added on the enclosure that dock to a charger using spring-loaded contacts.

Other form factors of a sensor snap may include, but are not limited to, jewelry, accessories, and attachables. Sensor fasteners can be used as a replacement for caps of snap fasteners. As a fastener replacement, a sensor fastener may look appropriate with fastener-heavy clothing. Some of the potential uses include jewelry such as bracelets and necklaces. A sensor fastener can also be placed on belts, shoes, zippers, and backpack straps. Beyond wearables, a sensor fastener can be placed on objects and in the environment, for example, to add sensing capabilities to toys. Bluetooth RSSI (received signal strength indication) can be used to track proximity of attachables, objects that have been attached with sensor snaps.

Further miniaturization of a sensor fastener can be provided. In one example, a sensor fastener has a physical size of 15 mm and 1 g. The size of the electronics can be reduced with a more dense layout. A sensor fastener can be integrated directly in buttons in some examples.

Various software and usability of a sensor fastener can be provided in accordance with example embodiments. In some implementations, there may only be one type of sensor fastener. In other implementations, there might be various sensor fasteners with different sensors and actuators. To efficiently manage the multiple sensors, a scalable software layer on a PC or mobile phone can configure, visualize, and control various sensor fasteners.

Power optimization can be provided with hardware and firmware modifications. For example, the battery can be optimized to last for at least a full day. The IMU can be replaced with a low power alternative. Some state-of-the-art IMUs consume as little as 0.55 mA with 3D fusion (e.g., LSM6DSOX, STMicro-electronics). Second, the capacitive touch library can be further optimized for low power. Currently, it can consume about 2 mA in some examples, which could be lowered by disabling the timers and ADC between the samples.

As demonstrated herein, it is feasible to integrate a wireless sensor node into fabric snap buttons. A screw mechanism or other attachment member can be provided that allows one to attach and detach sensor fasteners from off-the-shelf plastic snaps quickly. A 9-axis IMU can be used to sense tap, rotation, and orientation. Capacitive touch can be used to exit a standby mode. Potential applications of sensor fasteners include, but are not limited to, clothing augmentation for motion tracking applications. Dynamic power optimization is provided in some examples such that a sensor fastener can have a possible battery life of 4 hours in standby mode or 45-minute battery life in gesture mode. Other times may be provided

In some examples, a sensor fastener allows one to augment clothing with electronics and sensors quickly. Sensor fasteners can be potentially manufactured on a large scale. Sensor fasteners can pave the way for new interactive textiles that can be manipulated intuitively and integrate seamlessly, yet provide sophisticated sensing and communication capabilities.

By way of example, an interactive object can include a “soft” object such as a garment, garment accessory, or garment container at least partially formed from a flexible substrate. The flexible substrate may be formed of a soft material such as leather, natural fibers, synthetic fibers, or networks of such fibers. The flexible substrate may include a textile such as a woven or non-woven fabric, or other materials such as flexible plastics, films, etc. Materials may be formed by weaving, knitting, crocheting, knotting, pressing threads together or consolidating fibers or filaments together in a nonwoven manner. Interactive objects may also include “hard” objects such as may be made from nonflexible or semi-flexible materials such as plastic, metal, aluminum, and so on. A sensor fastener in accordance with embodiments of the present disclosure may be incorporated or otherwise applied to at least partially formed soft objects and/or hard objects.

Interactive objects can include “flexible” objects, such as a shirt, a hat, a handbag, and a shoe. It is to be noted, however, that a sensor fastener may be integrated with any type of flexible object made from fabric or a similar flexible material, such as garments or articles of clothing, garment accessories, garment containers, blankets, shower curtains, towels, sheets, bed spreads, or fabric casings of furniture, to name just a few. Examples of garment accessories may include sweat-wicking elastic bands to be worn around the head, wrist, or bicep. Other examples of garment accessories may be found in various wrist, arm, shoulder, knee, leg, and hip braces or compression sleeves. Headwear is another example of a garment accessory, e.g. sun visors, caps, and thermal balaclavas. Examples of garment containers may include waist or hip pouches, backpacks, handbags, satchels, hanging garment bags, and totes. Garment containers may be worn or carried by a user, as in the case of a backpack, or may hold their own weight, as in rolling luggage. A sensor fastener may be integrated within flexible objects in a variety of different ways, including weaving, sewing, gluing, and so forth.

Interactive objects may further include “hard” objects, such as a plastic cup and a hard smart phone casing. It is to be noted, however, that hard objects may include any type of “hard” or “rigid” object made from non-flexible or semi-flexible materials, such as plastic, metal, aluminum, and so on. For example, hard objects may also include plastic chairs, water bottles, plastic balls, or car parts, to name just a few. In another example, hard objects may also include garment accessories such as chest plates, helmets, goggles, shin guards, and elbow guards. Alternatively, the hard or semi-flexible garment accessory may be embodied by a shoe, cleat, boot, or sandal. A sensor fastener may be integrated within hard objects using a variety of different manufacturing processes.

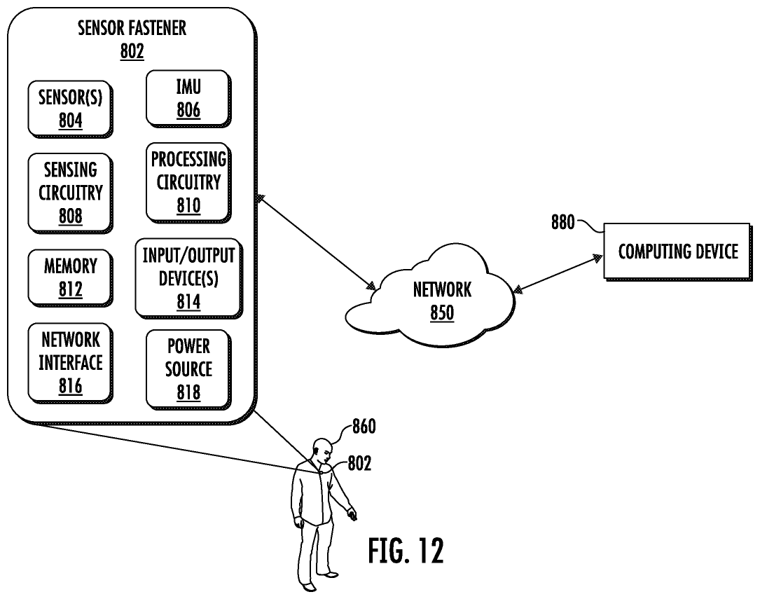

FIG. 12 illustrates an example environment 800 that includes a sensor fastener 802 that is capable of communication with one or more remote computing devices 880 over one or more networks 850. Sensor fastener 802 can include one or more sensors 804, one or more inertial measurement unit(s) 806 (IMUs), sensing circuitry 808, processing circuitry 810, memory 812 (RAM and/or ROM), input/output device(s) 814 (e.g., speakers, LEDs, microphones, touch sensors), network interface 816 (e.g., Bluetooth, WiFi, USB), and/or power source 818 (e.g., battery).

A sensor 804 can include a touch sensor such as a resistive or capacitive touch sensor. A touch sensor may include one or more sensing elements such as conductive threads or other sensing lines that are configured to detect a touch input. In some examples, a capacitive touch sensor can be formed from an interactive textile which is a textile that is configured to sense multi-touch-input. Textiles may be formed by weaving, knitting, crocheting, knotting, pressing threads together or consolidating fibers or filaments together in a nonwoven manner. A capacitive touch sensor can be formed from any suitable conductive material and in other manners, such as by using flexible conductive lines including metal lines, filaments, etc. attached to a non-woven substrate. Other types of sensors such as strain gauges, ultrasonic sensors, radar-based touch interfaces, image-based sensors, infrared sensors, etc. can be integrated within a sensor fastener as described.

One type of sensor includes an inertial measurement unit(s) 806 (IMU(s)) which can generate sensor data indicative of a position, velocity, and/or an acceleration of the interactive object. The IMU(s) 806 may generate one or more outputs describing one or more three-dimensional motions of the sensor fastener 802. The IMU(s) may be secured to a printed circuit board, for example, with zero degrees of freedom, either removably or irremovably, such that the inertial measurement unit translates and is reoriented as the sensor fastener into two is translated and are reoriented. In some embodiments, the inertial measurement unit(s) 806 may include a gyroscope or an accelerometer (e.g., a combination of a gyroscope and an accelerometer), such as a three axis gyroscope or accelerometer configured to sense rotation and acceleration along and about three, generally orthogonal axes. In some embodiments, the inertial measurement unit(s) may include a sensor configured to detect changes in velocity or changes in rotational velocity of the interactive object and an integrator configured to integrate signals from the sensor such that a net movement may be calculated, for instance by a processor of the inertial measurement unit, based on an integrated movement about or along each of a plurality of axes.

In environment 800, the electronic components contained within the sensor fastener 802 also include sensing circuitry 808 that is coupled to sensor(s) to generate one or more outputs indicative of an input detected by a sensor 804. Power source 818 may be coupled, via one or more interfaces to provide power to the various components of the sensor fastener 802, and may be implemented as a small battery in some examples. Power source 818 may be coupled to sensing circuitry 808 to provide power to sensing circuitry 808 to enable the detection of input via sensor(s) 804. Power source 818 can be removable or embedded within a wearable device in example embodiments. Sensing circuitry 808 can include various components such as amplifiers, filters, charging circuits, sense nodes, and the like that are configured to sense one or more electrical characteristics of a user via a sensor 804. Sensing circuitry 808 can be implemented as voltage sensing circuitry, current sensing circuitry, capacitive sensing circuitry, resistive sensing circuitry, etc. In some examples, sensing circuitry 808 can generate one or more signals that are representative of one or more inputs such as motion, touch, etc. detected by a sensor 804.

Processing circuitry 810 can include one or more electric circuits that comprise one or more processors such as one or more microprocessors. Memory 812 can include (e.g., store, and/or the like) instructions. When executed by processing circuitry 810, instructions stored in memory 812 can cause processing circuitry 810 to perform one or more operations, functions, and/or the like described herein. Processing circuitry can analyze a sensor signal or other electrical characteristic in order to determine data indicative of an input measure by the sensor of the user. By way of example, processing circuitry 810 can generate data indicative of gestures, metrics, heuristics, trends, predictions, or other measurements associated with the sensor fastener.

Sensor fastener 802 may include one or more input/output devices 814. An input device such as a touch input device can be utilized to enable user to provide input to the wearable device. An output device can be configured to provide a haptic response, a tactical response, an audio response, a visual response, or some combination thereof. Output devices may include visual output devices, such as one or more light-emitting diodes (LEDs), audio output devices such as one or more speakers, one or more tactile output devices, and/or one or more haptic output devices. In some examples, the one or more output devices are formed as part of the sensor fastener, although this is not required. In one example, an output device can include one or more LEDs configured to provide different types of output signals. For example, the one or more LEDs can be configured to generate patterns of light, such as by controlling the order and/or timing of individual LED activations based on inputs detected by one or more sensors. Other lights and techniques may be used to generate visual patterns including circular patterns. In some examples, one or more LEDs may produce different colored light to provide different types of visual indications. Output devices may include a haptic or tactile output device that provides different types of output signals in the form of different vibrations and/or vibration patterns. In yet another example, output devices may include a haptic output device such as may tighten or loosen an interactive object with respect to a user. For example, a clamp, clasp, cuff, pleat, pleat actuator, band (e.g., contraction band), or other device may be used to adjust the fit of a wearable device on a user (e.g., tighten and/or loosen).

Network interface 816 can enable sensor fastener 802 to communicate with one or more computing devices 880. By way of example and not limitation, network interfaces 816 may communicate data over a local-area-network (LAN), a wireless local-area-network (WLAN), a personal-area-network (PAN) (e.g., Bluetooth™), a wide-area-network (WAN), an intranet, the Internet, a peer-to-peer network, point-to-point network, a mesh network, and the like. Network interface 816 can be a wired and/or wireless network interface.

By way of example, sensor fastener 802 may transmit data indicative of a sensor input to one or more remote computing devices in example embodiments. By way of example, when a gesture or touch input is detected by sensing circuitry 808 and/or processing circuitry 810 of the sensor fastener, data representative of the gesture or touch input may be communicated, via network interface 816, to a remote computing device 880 via network 850. In some examples, one or more outputs of sensing circuitry 808 are received by a microprocessor of processing circuitry 810. The microprocessor may then analyze the output of the sensing circuitry (e.g., a sensor signal) to determine data associated with a sensor input. The data and/or one or more control signals may then be communicated to a computing device 880 (e.g., a smart phone, server, cloud computing infrastructure, etc.) via the network interface 850 to cause the computing device to initiate a particular functionality. Generally, network interfaces 816 are configured to communicate data, such as sensor data, over wired, wireless, or optical networks to computing devices.

In some examples, the internal electronics of the wearable device sensor fastener 802 can include a flexible printed circuit board (PCB). The printed circuit board can include a set of contact pads for attaching to a sensor 804. In some examples, one or more of sensing circuitry 808, processing circuitry 810, input/output devices 814, memory 812, power source 818, and network interface 816 can be integrated on the flexible PCB.

Sensor fastener 802 can include various other types of electronics, such as additional sensors (e.g., capacitive touch sensors, microphones, accelerometers), output devices (e.g., LEDs, speakers, or micro-displays), electrical circuitry, and so forth. The various electronics depicted within sensor fastener 802 may be physically and permanently embedded within sensor fastener 802 in example embodiments. In some examples, one or more components may be removably coupled to the sensor fastener. By way of example, a removable power source 818 may be included in example embodiments.

While sensor fastener 802 is illustrated and described as including specific electronic components, it will be appreciated that interactive object such as wearable devices may be configured in a variety of different ways. For example, in some cases, electronic components described as being contained within an interactive object may at least be partially implemented at another computing device, and vice versa. Furthermore, sensor fastener 802 may include electronic components other that those illustrated in FIG. 8, such as sensors, light sources (e.g., LED's), displays, speakers, and so forth.

Sensor fastener 802 is one example of a wearable device category of interactive objects as described herein. It will be appreciated that while specific components are depicted in FIG. 15, additional or fewer components may be included in an interactive object in accordance with example embodiments of the present disclosure.

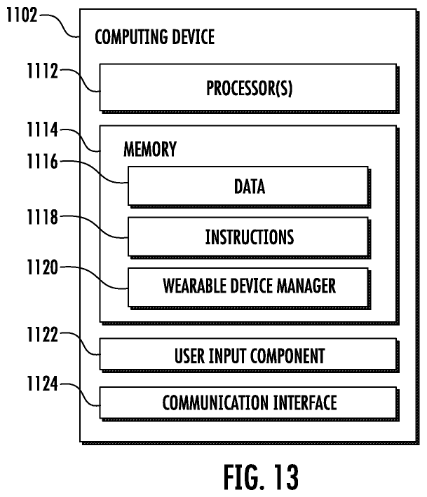

FIG. 13 illustrates various components of an example computing system 1102 that can implement any type of client, server, wearable, and/or other computing device described herein. In embodiments, computing system 1102 can be implemented as one or a combination of a wired and/or wireless wearable device, System-on-Chip (SoC), and/or as another type of device or portion thereof. Computing system 1102 may also be associated with a user (e.g., a person) and/or an entity that operates the device such that a device describes logical devices that include users, software, firmware, and/or a combination of devices. By way of example, computing system 1102 can be used to implement a wearable device or computing device such as computing device 880 as described herein. The computing system 1102 can be a user computing device which can include, for example, a personal computing device (e.g., laptop or desktop), a mobile computing device (e.g., smartphone or tablet), a gaming console or controller, a wearable computing device, an embedded computing device, or any other type of computing device.

The computing system 1102 includes one or more processors 1112 and a memory 1114. The one or more processors 1112 can be any suitable processing device (e.g., a processor core, a microprocessor, an ASIC, an FPGA, a controller, a microcontroller, etc.) and can be one processor or a plurality of processors that are operatively connected. The one or more processors can process various computer-executable instructions to control the operation of computing system 1102 and to enable techniques for, or in which can be embodied a wearable device. Alternatively or in addition, computing system 1102 can be implemented with any one or combination of hardware, firmware, or fixed logic circuitry that is implemented in connection with processing and control circuits. Although not shown, computing system 1102 can include a system bus or data transfer system that couples the various components within the device. A system bus can include any one or combination of different bus structures, such as a memory bus or memory controller, a peripheral bus, a universal serial bus, and/or a processor or local bus that utilizes any of a variety of bus architectures.

The memory 1114 can include one or more non-transitory computer-readable storage mediums, such as RAM, ROM, EEPROM, EPROM, flash memory devices, magnetic disks, etc., and combinations thereof. The memory 1114 can store data 1116 and instructions 1118 which are executed by the processor 1112 to cause the user computing system 1102 to perform operations. Memory 1114 enables persistent and/or non-transitory data storage (i.e., in contrast to mere signal transmission). A disk storage device may be implemented as any type of magnetic or optical storage device, such as a hard disk drive, a recordable and/or rewriteable compact disc (CD), any type of a digital versatile disc (DVD), and the like. Memory 1114 may also include a mass storage media device of computing system 1102.

Computing system 1102 includes a communication interface 1124 that enables wired and/or wireless communication of data 1116 (e.g., received data, data that is being received, data scheduled for broadcast, data packets of the data, etc.). Data 1116 can include configuration settings of the device, media content stored on the device, and/or information associated with a user of the device. Media content stored on computing system 1102 can include any type of audio, video, and/or image data. Computing system 1102 includes one or more data inputs via which any type of data, media content, and/or inputs can be received, such as human utterances, touch data generated by a touch sensor, user-selectable inputs (explicit or implicit), messages, music, television media content, recorded video content, and any other type of audio, video, and/or image data received from any content and/or data source.

Communication interfaces can be implemented as any one or more of a serial and/or parallel interface, a wireless interface, any type of network interface, a modem, and as any other type of communication interface. Communication interfaces provide a connection and/or communication links between computing system 1102 and a communication network by which other electronic, computing, and communication devices communicate data with computing system 1102.

Computer-readable media provides data storage mechanisms to store device data, as well as computer-readable instructions 1118 which can implement various device applications and any other types of information and/or data related to operational aspects of computing system 1102. For example, an operating system can be maintained as a computer application with computer-readable media and executed on processors 1112. Device applications may include a device manager, such as any form of a control application, software application, signal-processing and control module, code that is native to a particular device, a hardware abstraction layer for a particular device, and so on.

Memory 1114 may also include a wearable device manager 1120. Wearable device manager 1120 is capable of interacting with applications and a remote wearable device effective to activate various functionalities associated with computing system 1102 and/or applications through input received from a wearable device. Wearable device manager 1120 may be implemented at a computing device that is local to the wearable device or remote from the wearable device. Wearable device manager 1120 is one example of a controller. In some implementations, wearable device manager 1120 may include one or more sensor component(s). For example, a sensor component can store and/or analyze sensor data from a wearable device. In some examples, sensor component can generate data associated with sensor-detected activity, inputs, etc. In some examples, the wearable device manager 1120 can include detection, classification, prediction or other models that can generate inferences based on sensor data associated with a wearable device. In some examples, the wearable device manager 1120 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks. The wearable device manager 1120 can be stored in the user computing device memory 1114, and then used or otherwise implemented by the one or more processors 1112.

The computing system 1102 can also include one or more user input components 1122 that receive user input. For example, the user input component 1122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

In some implementations, the computing system 1102 includes or is otherwise implemented by a computing system including one or more computing devices. In instances in which the computing system 1102 is implemented as part of plural server computing devices, such computing devices can operate according to sequential computing architectures, parallel computing architectures, or some combination thereof.

FIG. 13 illustrates one example computing system that can be used to implement the present disclosure. Other computing systems can be used as well.

The technology discussed herein makes reference to servers, databases, software applications, and other computer-based systems, as well as actions taken and information sent to and from such systems. One of ordinary skill in the art will recognize that the inherent flexibility of computer-based systems allows for a great variety of possible configurations, combinations, and divisions of tasks and functionality between and among components. For instance, server processes discussed herein may be implemented using a single server or multiple servers working in combination. Databases and applications may be implemented on a single system or distributed across multiple systems. Distributed components may operate sequentially or in parallel.

In some implementations, in order to obtain the benefits of the techniques described herein, the user may be required to allow the collection and analysis of sensor data or other personal information associated with the user or their device. For example, in some implementations, users may be provided with an opportunity to control whether programs or features collect such information. If the user does not allow collection and use of such signals, then the user may not receive the benefits of the techniques described herein. The user can also be provided with tools to revoke or modify consent. In addition, certain information or data can be treated in one or more ways before it is stored or used, so that personally identifiable information is removed. As an example, a computing system can obtain real-time personal data which can indicate attributes of a user, without identifying any particular user(s) or particular user computing device(s).

While the present subject matter has been described in detail with respect to specific example embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. A sensor fastener, comprising:
a first fastener component;
a second fastener component comprising:
a housing;
an inertial measurement unit disposed within the housing;
processing circuitry disposed within the housing and communicatively coupled to the inertial measurement unit; and
at least one attachment member that physically couples the housing to at least the first fastener component; and
a third fastener component releasably coupled with the first fastener component to releasably fasten at least two portions of an interactive object to one another.

2. The sensor fastener of claim 1, wherein:
the first fastener component implements a socket of the sensor fastener;
the second fastener component implements a cap of the sensor fastener;
the third fastener component implements a post of the sensor fastener; and
the socket and the post releasably snap together and thereby releasably fasten the at least two portions of the interactive object to one another.

3. The sensor fastener of claim 2, wherein:
the cap includes at least one receiving member to receive the at least one attachment member; and
the at least one attachment member is configured to pass through at least a portion of the interactive object and physically couple to the at least one receiving member to thereby securably attach the sensor fastener to the interactive object.

4. The sensor fastener of claim 3, wherein:
the housing includes a base and a lid;
the cap comprises a printed circuit board including the inertial measurement unit and the processing circuitry, the printed circuit board is coupled to the base; and
the base includes an opening through which the at least one attachment member physically couples to the at least one receiving member to securably attach the sensor fastener to the interactive object.

5. The sensor fastener of claim 4, further comprising:
a capacitive touch sensor;
wherein the printed circuit board includes sensing circuitry communicatively coupled to the capacitive touch sensor.

6. The sensor fastener of claim 5, wherein:
the capacitive touch sensor is physically coupled to an inside surface of the lid of the housing.

7. The sensor fastener of claim 1, wherein:
the at least one attachment member includes a screw.

8. The sensor fastener of claim 1, wherein:
the housing includes one or more attachment features forming at least a portion of the at least one attachment member.

9. The sensor fastener of claim 1, wherein:
the first fastener component implements a stud of the sensor fastener;
the second fastener component implements a post of the sensor fastener;
the third fastener component implements a socket of the sensor fastener; and
the socket and the post releasably snap together and thereby releasably fasten the at least two portions of the interactive object to one another.

10. The sensor fastener of claim 1, wherein:
the interactive object comprises a wearable garment including a textile substrate; and
the sensor fastener is a snap sensor fastener.

11. A sensor fastener, comprising:
a post;
a stud configured to pass through a flexible substrate of an interactive object and physically attach to the post;
a socket releasably and physically coupled to the post; and
a cap, comprising:
a housing;
an inertial measurement unit disposed within the housing;
processing circuitry disposed within the housing and communicatively coupled to the inertial measurement unit; and
at least one attachment member configured to pass through the flexible substrate and physically couple the housing to the socket of the sensor fastener.

12. The sensor fastener of claim 11, wherein:
the cap includes at least one receiving member to receive the at least one attachment member and thereby physically couple the housing to the socket of the sensor fastener.

13. The sensor fastener of claim 11, wherein:
the housing includes a base and a lid;
the cap comprises a printed circuit board including the inertial measurement unit and the processing circuitry, the printed circuit board is coupled to the base; and
the base includes an opening through which the at least one attachment member physically couples to the at least one receiving member to securably attach the sensor fastener to the interactive object.

14. The sensor fastener of claim 13, further comprising:
a capacitive touch sensor;
wherein the printed circuit board includes sensing circuitry communicatively coupled to the capacitive touch sensor.

15. The sensor fastener of claim 14, wherein:
the capacitive touch sensor is physically coupled to an inside surface of the lid of the housing.

16. An interactive object, comprising:
at least one substrate; and
at least one sensor fastener physically and removably coupled to the at least one substrate, the sensor fastener comprising a post, a stud that passes through the at least one substrate and physically couples to the post, a socket releasably and physically coupled to the post, and a cap, wherein the cap comprises:
a housing;
an inertial measurement unit disposed within the housing;
processing circuitry disposed within the housing and communicatively coupled to the inertial measurement unit; and
at least one attachment member that passes through the at least one substrate and physically couples the housing to the socket.

17. The interactive object of claim 16, wherein the interactive object is at least one of a garment, garment accessory, or garment container.

18. The interactive object of claim 16, wherein:
the cap includes at least one receiving member to receive the at least one attachment member and thereby physically couple the housing to the socket of the sensor fastener.

19. The interactive object of claim 16, wherein:
the housing includes a base and a lid;
the cap comprises a printed circuit board including the inertial measurement unit and the processing circuitry, the printed circuit board is coupled to the base; and
the base includes an opening through which the at least one attachment member physically couples to the at least one receiving member to securably attach the sensor fastener to the interactive object.

20. The interactive object of claim 19, further comprising:
a capacitive touch sensor;
wherein the printed circuit board includes sensing circuitry communicatively coupled to the capacitive touch sensor.
